(19) United States
(12) Patent Application Publication
(10) Pub. No.: US 2005/0171494 A1
(43) Pub. Date: Aug. 4, 2005

(54) Aseptic eyedropper and method for its use

(76) Inventor: Jonathan H. Cress, Santa Cruz, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/099,322

(22) Filed: Apr. 4, 2005

Related U.S. Application Data
(63) Continuation-in-part of application No. PCT/US04/27585, filed on Aug. 24, 2004.
Application No. PCT/US04/27585 is a continuation-in-part of application No. 10/648,138, filed on Aug. 25, 2003.

(51) Int. Cl. A61M 35/00

(52) U.S. Cl. 604/300

(57) ABSTRACT

Apparatus for attaching to a squeeze bottle or other dispenser for delivering drops to an eye comprises an axial member and a laterally extending leg. The axial member is attached to the bottle and receives drops or liquid therefrom. The leg is rested against the patient's nose bridge so that it releases drops into the eye, preferably a corner of the eye.

### CROSS-REFERENCES TO RELATED APPLICATIONS

This application is a continuation-in-part of PCT/JUS04/27585 (Attorney Docket No. 012993-000610PC), filed on Aug. 24, 2004, which was a continuation-in-part of U.S. application Ser. No. 10/648,138, filed on Aug. 25, 2003, the full disclosures of which are incorporated herein by reference.

### BACKGROUND OF THE INVENTION

1. Field of the Invention

The present invention relates generally to medical apparatus and methods. More particularly, the present invention relates to eyedropper construction and methods for using eyedroppers to deliver drops to the inner corner of a patient's eye.

Many if not most people find dispensing eye drops into their own eyes to be an uncomfortable procedure. Many people blink or shake as the drops are released from the conventional eyedropper, often leading to incomplete delivery of the irrigant lubricant or medication.

Despite many attempts to provide improved systems, the most common technique for self-dispensation of eye drops remains use of a dropper bottle. The patient turns his or her eyes upwardly, locates a dispensing nozzle of the dropper over the eye, and squeezes the bottle to dispense the drop. Sometimes this works. Sometimes this doesn't work.

Prior attempts at improving eyedroppers and systems are almost too numerous to mention. Of particular interest to the present invention, a variety of eye drop dispensing guides or positioners have been developed. For the most part, these improved guides or dispensers have been attempts to stabilize and/or position the dispensing nozzle of the dropper or dropper bottle at some predetermined position over the eye. In some cases, the guides or positioners have further included means for holding the eyelid open in an attempt to assure that the drop actually enters the eye.

While potentially being helpful, the location of the dropper or dropper bottle over the eye still can be disturbing or even daunting to the patient. Moreover, most of these guides or positioners have been threatening, somewhat uncomfortable, cumbersome, unaesthetic, and awkward to use.

For these reasons, it would be desirable to provide additional and improved devices and apparatus for accurately and successfully dispensing drops into a patient's eyes. In particular, it would be desirable if the apparatus and systems were easy to use, did not require placement of the dispensing bottle or dropper directly over an eye, and could provide accurate and reproducible delivery of a drop to a particular area of the eye, more particularly the inner corner (medial canthus) of the eye. At least some of these objectives will be met by the invention as described and claimed hereinbelow.

2. Description of the Background Art

Patents describing eyedroppers and other devices for directing drops into eyes include U.S. Pat. Nos. 6,595,970; 6,325,784; 6,319,240; 5,713,495; 5,665,079; 5,059,188; 4,973,322; 4,960,407; 4,685,906; 4,468,103; 4,257,417; 4,085,750; and 3,934,590. See also published application U.S. 2002/0016576 and design patents D463,550; D457,952; and D369,211. The full disclosures of each of these patents and applications are incorporated herein by reference.

### BRIEF SUMMARY OF THE INVENTION

The present invention provides drop dispensing apparatus and methods intended particularly for delivering drops to an eye from a generally conventional drop applicator, such as a squeeze bottle. In particular, the apparatus comprises a drop dispensing attachment which is adapted to receive drops from such a squeeze bottle and to direct the drops into a patient's eye, particularly to the inner corner of the eye, i.e., the medial canthus. The drop dispensing attachment may be provided as a separate component which is capable of being attached to an otherwise conventional drop dispensing squeeze bottle. Alternatively, the attachment may be formed as an integral part of such a squeeze bottle or other dispenser and/or may include a special connector which allows the attachment to be removably attached to squeeze bottles or other dispensers having a special mating attachment.

Droplet dispensing attachments according to the present invention comprise an axial member having an inlet which receives drops from the squeeze bottle and a cross member attached to the axial member. The cross member includes a nose bridge rest which is adapted to rest on the patient's nose bridge. In exemplary embodiments, the nose bridge rest is axially aligned with the axial member and the attached squeeze bottle so that the patient is able to hold the squeeze bottle and advance the nose rest over the patient's own nose bridge so that the bottle is between the eyes, not directly over either eye.

The cross member of the dispensing attachment further includes at least one leg extending laterally relative to the axial member (usually the nose bridge rest as well). The laterally extending leg has an outlet which is connected to receive drops from the inlet in the axial member. The outlet is further adapted and positioned so that it will release drops from the outlet into the patient's eye when the rest is positioned over the patient's nose bridge. In the preferred and exemplary embodiments, the outlet will be positioned so that the drop is released into the inner corner of the eye. In this way, there is little or no structure which is positioned directly over the patient's eye, significantly reducing the negative perception which many patients feel when placing an eyedropper over their eye.

In a first exemplary embodiment, the cross member will have an arcuate shape with two generally symmetric legs wherein the nose bridge rest is disposed between the legs. One of the legs will provide the lateral extending leg for dispensing the drops, while the other leg may be shorter or truncated so that its primary purpose is to define the symmetric nose bridge which is placed over the patient's nose.

In further exemplary embodiments, the outlet in the laterally extending leg will terminate in a nozzle which provides the actual release of the drops being delivered. Still more preferably, the nozzle will be protected within a recess formed in the end of the laterally extending leg, helping to assure that the nozzle remains clean and assuring that the nozzle does not directly contact the eye.

The nozzle within the recess may be oriented in a variety of different ways in order to release the drop directly or indirectly into the eye. In particular, when the axial member is held generally vertically against the patient's nose bridge, the nozzle may be positioned directly above the eye to release the drop directly into the eye. Alternatively, when the axial member is held generally vertically against the nose bridge, the nozzle may be oriented to release the drop onto an edge of the recess, where the drop then flows or drops off the edge indirectly into the eye. In the latter case, it may be desirable to treat the surface of the recess which receives the drop so that it is hydrophobic and promotes release of the drop into the eye. In the embodiments where the drop is going directly into the eye, the recess may have at least one cut-out portion disposed on an inner side to reduce contact between the edge of the recess and the drop as it is released. Often, there will be at least two such cut-outs which help prevent pooling of the droplets within the recess and also provide for venting of the area within the recess as the drop is dispensed. In a particularly preferred embodiment, the nozzle can be located over an opening or aperture in the bottom of the recess to allow the drop to fall with a minimum chance of contacting the edge of the recess.

In still further embodiments of the present invention, the attachment is provided as an integral or removable part of a system which further includes the squeeze bottle which carries the desired fluid to be delivered to the eye, typically being an irrigant, a lubricant, a medication, or a combination thereof. The attachment and/or complete systems will typically be provided in sterile packages for delivery to patients.

According to a further aspect of the present invention, a method for dispensing drops into the eye of a patient comprises aligning a squeeze bottle axially with the bridge of the patient's nose, i.e., so that it is positioned generally between the patient's eye and not aligned over either eye. A drop is dispensed from the bottle in an axial direction toward the bridge of the patient's nose. The drop is then laterally channeled away from the axis so that it is released into the eye, preferably into the inner corner of the eye adjacent to the patient's nose bridge. Aligning typically comprises resting a cross member attached to the squeeze bottle on the patient's nose bridge. Laterally channeling typically comprises diverting the drop through a lumen in the cross member, where a terminal end of the lumen, typically formed as a nozzle, is disposed, over the eye, preferably over the inner corner of the eye.

The methods of the present invention can be performed to release the drop directly into the eye by holding the nozzle directly over the eye when the drop is released. In such cases, the edge of the recess will be positioned away from the path of the drop as it falls to the eye. Usually, the edge of the recess will be disposed directly against the corner of the eye, often with a cut-out disposed at the corner to prevent pooling of the droplet and optionally vent the area within the recess. Alternatively, the nozzle can be oriented so that the droplet falls on an edge of the recess and flows indirectly into the eye.

### BRIEF DESCRIPTION OF THE DRAWINGS

FIG. 1 is a side view of a drop dispensing attachment constructed in accordance with the principles of the present invention.

FIG. 2 is a cross-sectional view of the drop dispensing attachment of FIG. 1.

FIG. 3 is an end view of a laterally extending leg of a cross-member of the drop dispensing apparatus of FIGS. 1 and 2 taken along line 3-3 of FIG. 2.

FIGS. 4 and 5 illustrate use of the apparatus of FIGS. 1-3 for dispensing drops into an inner corner of a patient's eye.

FIG. 6 is a perspective view of an alternative drop dispensing system constructed in accordance with the principles of the present invention.

FIG. 7 is an exploded view of the system of FIG. 6, shown with the attachment in cross-section.

FIG. 8 is a side-view of the attachment showing a cross-sectional line 7-7.

FIG. 9 illustrates use of the device of FIG. 6 for dispensing a drop directly into an eye.

FIGS. 10 and 11 illustrate a further alternative structure for a nozzle arrangement according to the present invention.

FIGS. 12A and 12B illustrate use of the device of FIGS. 9 and 10 for dispensing drops at different head inclinations.

### DETAILED DESCRIPTION OF THE INVENTION

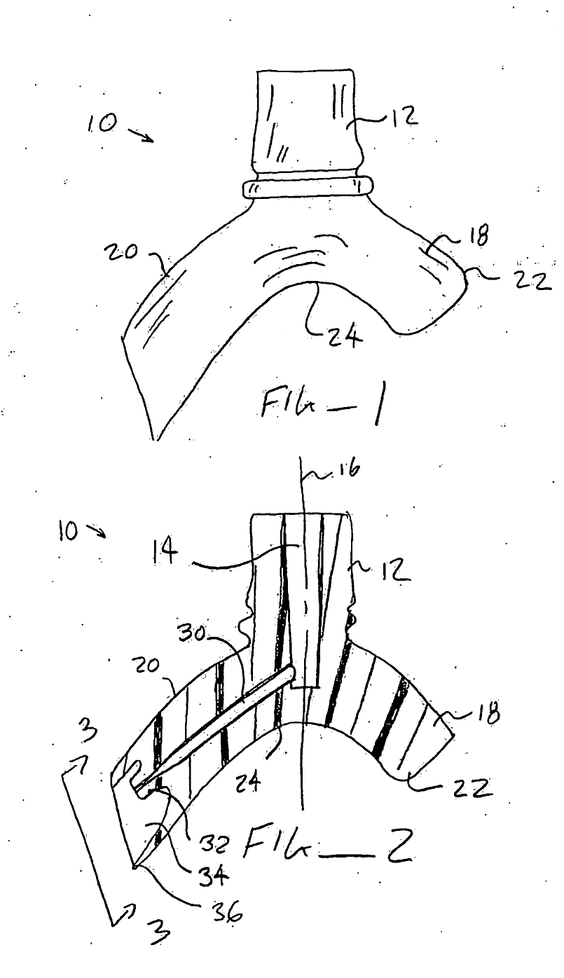

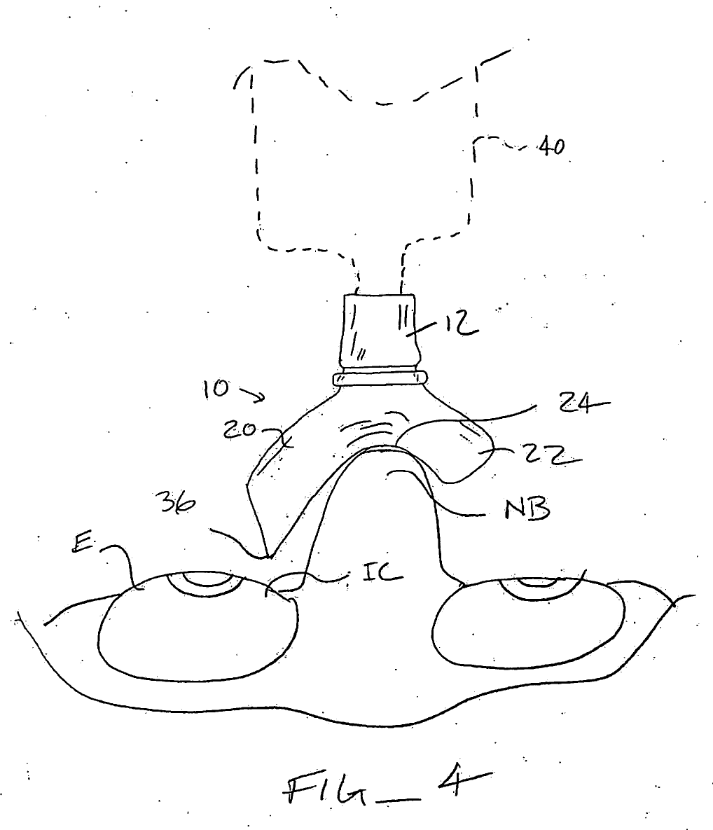

An exemplary drop dispensing attachment 10 constructed in accordance with the principles of the present invention is illustrated in FIGS. 1-3. The attachment 10 includes an axial member 12 having an inlet lumen 14 aligned generally along a longitudinal axis 16 of the device. A cross member 18 is attached to the axial member 12 and includes at least one laterally extending leg 20 and usually a second laterally extending leg 22. Preferably, the legs 20 and 22 are arranged in an arcuate geometry to define a nose bridge rest region 24 therebetween. The laterally extending leg 20 includes an outlet lumen 30 which preferably terminates in a nozzle 32 found within a recess 34 in the end of the leg 20. Preferably, the recess 34 terminates in a very thin circumferential segment or “lip” 36 at its lower end which will be positioned at the medial canthus or inner corner of the eye during the dispensing procedure, as described in more detail below. The thin lip 36 is desirable since it puts the drop in contact with the skin, thereby drawing it away from the device by capillary action and reducing the possibility of back contamination of the device and the bottle's content.

The axial member 12 will be adapted to be removably (or in some cases permanently) attached to a squeeze bottle or other droplet dispenser. Although not illustrated in detail, the axial member may be threaded, provided with an interference fit, or otherwise specially adapted to removably receive the squeeze bottle. Alternatively, of course, the attachment 10 could be formed as an integral part of the squeeze bottle or other droplet dispenser.

The attachment 10 may be formed from a variety of materials, typically being cast or molded from a suitable polymer such as polycarbonate, or silicone. The dimensions are chosen to fit a typical patient, with the lumens having diameters from 0.25 mm to 1 mm, the nozzle diameter being from 0.25 mm to 1 mm, and the distance from the axis 16 to the lip 36 being 15 mm to 25 mm. The materials are preferably malleable and conform to a variety of nasal bridge contours.

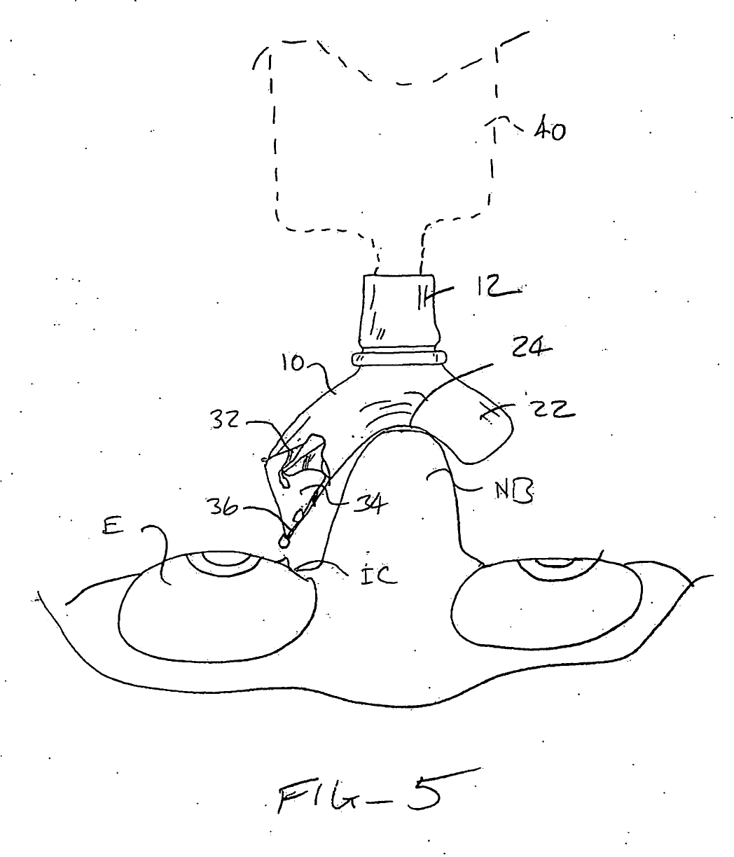

Referring now to FIGS. 4 and 5, use of the attachment 10 attached to a dispensing bottle 40 for delivering drops to an eye E of a patient will be described. Initially, the patient attaches the attachment 10 to the bottle 40, if it has not already been done. After that, the patient may hold the bottle, or in some cases less desirably the attachment 10, in order to engage the nose bridge region 24 of the attachment against the nose bridge NB of the patient. The assembly of the attachment 10 and squeeze bottle 40 will be held so that the lip 36 of the leg 20 is positioned over the inner corner IC of the eye E. Proper positioning of the assembly will generally be as shown in FIG. 4, where the assembly is generally between the eyes rather than over either eye.

By squeezing the bottle 40, droplets are dispensed through the attachment 10 and out of the nozzle 32, as illustrated in FIG. 5. The droplets will fall onto the inner wall of the recess 34 and then from the lip 36 and slide into the eye.

Conveniently, if the patient wishes to dispense drops into the other eye, the assembly of the bottle 40 and attachment 10 may simply be turned 180° about the axis 16 so that the dispensing leg 20 now lies over the other eye. The bottle may then be squeezed to deliver lubricant, medication, and/or irrigant to the other eye as just described with respect to the first eye.

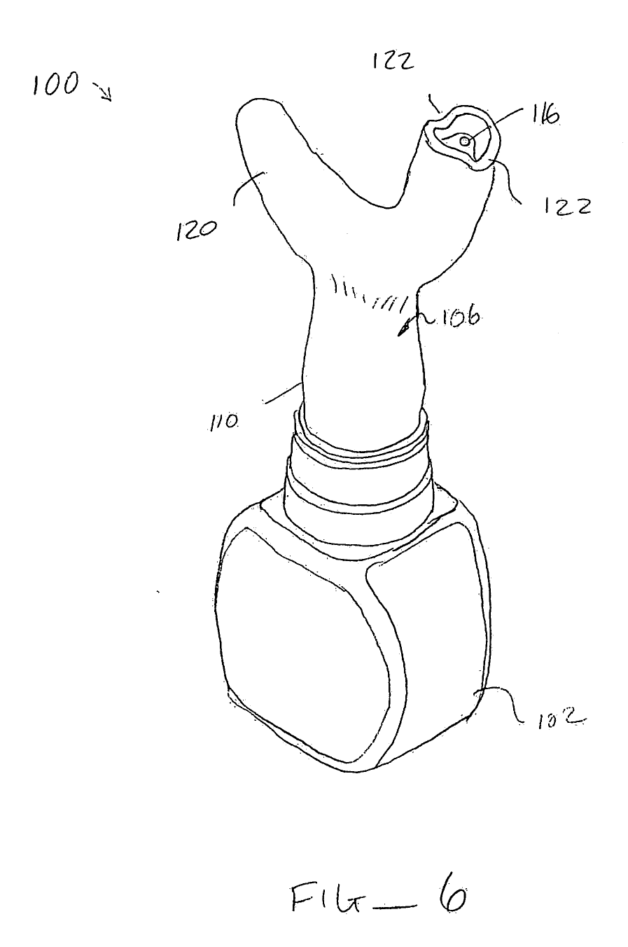

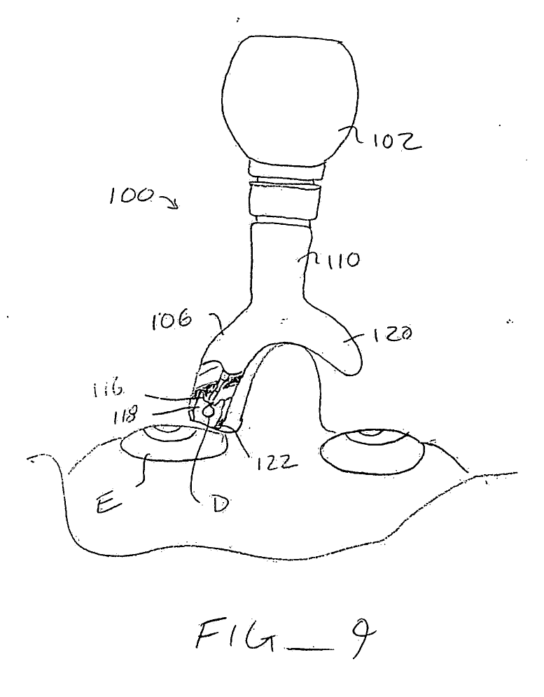

A drop dispensing system 100 constructed in accordance with the principles of the present invention and comprising a dropper bottle 102, a compression ring 104, and an attachment 106 for laterally deflecting the eye drop is illustrated in FIGS. 6-8. The dropper bottle 102 can be any conventional eye dropper having a tip 108 intended to dispense a drop in a conventional manner. The attachment 106 is a modified version of the attachments described earlier, as will be described in more detail below. The compression ring 104 is adapted to permit removable placement of the attachment 106 on the tip 108. Thus, it will be appreciated that an axial member 110 and the compression ring 104 can be modified and constructed as appropriate to allow removable attachment to any type of eye dropper bottle available to be modified. The materials and general dimensions of the attachment 106 may be similar or identical to those described earlier for attachment 10. The principal differences between attachment 106 and the prior attachment 110 is that the laterally extending leg 112 which carries the outlet lumen 114 and nozzle 116 extends over a greater arc length than that of the prior embodiment. As will be described in more detail with respect to FIG. 9 below, this modified configuration allows the nozzle 116 to dispense a drop directly into the eye without the drop first landing on any portion of the peripheral wall of recess 118. The attachment 106 will typically comprise a second leg 120 to complete the cross-member.

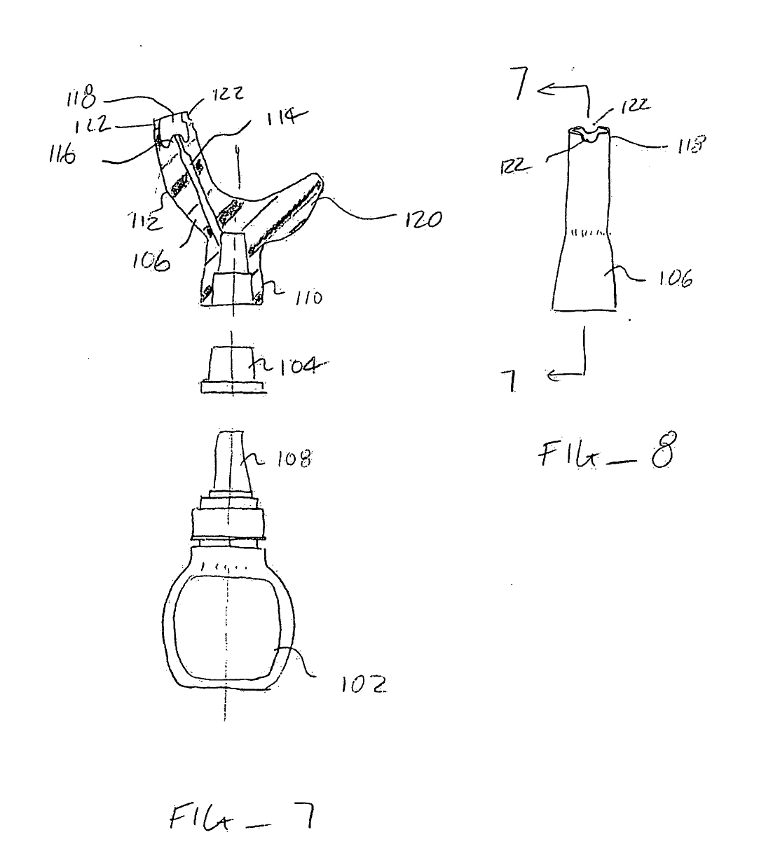

The attachment 106 further differentiates the earlier attachment in that a pair of cut-outs 122 are formed in the peripheral wall about recess 118, as best seen in FIG. 8. These cut-outs help assure that the drop will not contact the peripheral wall and further provide venting of the recessed region when the device is in use dispensing a drop into the eye.

Referring now to FIG. 9, the dropper bottle system 100 is used by placing the middle of the cross-member against the patient's nose bridge so that the lateral arm 116 is generally over the inner corner of the patient's eye E. The axis of axial member 110 will be held vertically and the nozzle 116 will be able to dispense a drop D directly into the corner of the eye, while the cut-out and remaining peripheral wall around the recess 118 contacts the inner portion of the eye or tissue surrounding the eye. Cut-out 122 helps prevent pooling of the droplet and helps vent the region within the recess, although presence of the cut-out is not essential for proper operation of the device.

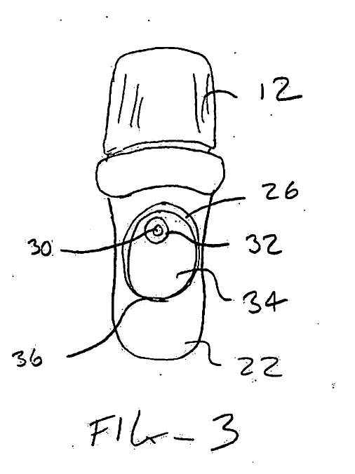

FIG. 10 illustrates an attachment 200 which has a geometry very similar to that of attachment 10 (FIGS. 1 and 2), except that a large cut-out 202 is formed in the lower lip 36 of the attachment. All other numbering in FIG. 10 will be the same as in FIGS. 1 and 2.

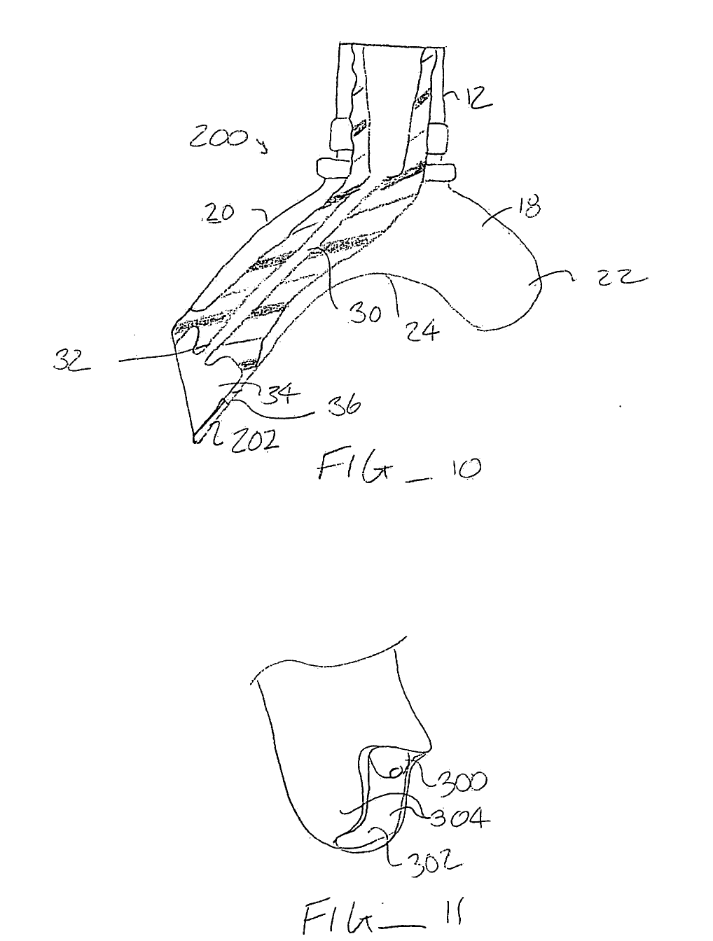

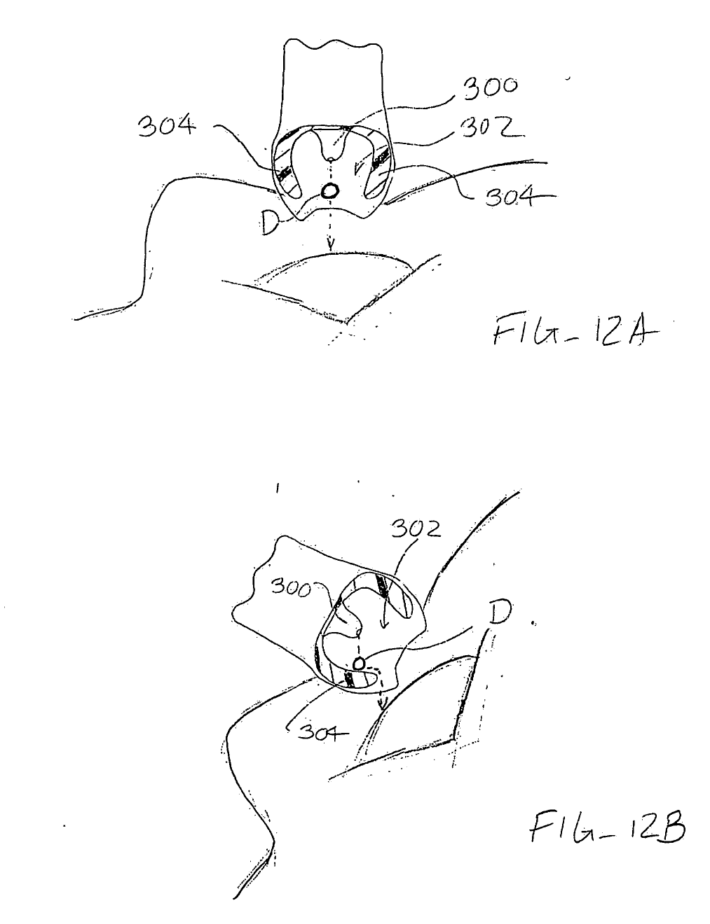

A still further embodiment of a nozzle 300 oriented vertically over an opening or aperture 302 in a cross-member 318 is illustrated in FIG. 11. The opening 302 of the nozzle 300 is defined by inwardly flared walls 304. The nozzle 300 will release a drop D directly over the eye with little risk of contacting an edge of the cross-member, thus significantly reducing any opportunity for contamination of the device or bottle contents, as illustrated in FIG. 12A where the patient has fully tilted his or her head. The inwardly flared side walls 304 will act to redirect the drop D to the medial canthal or inner corner of the eye even when the head is not inclined fully back, as illustrated in FIG. 12B.

While the invention has been described with reference to specific embodiments, the description is illustrative of the invention and is not to be construed as limiting the invention. Various modifications and applications may occur to those skilled in the art without departing from the true spirit and scope of the invention as defined by the appended claims.

1. A drop dispensing attachment adapted to receive drops from a squeeze bottle and to direct drops to an eye of a patient, said attachment comprising:
an axial member having an inlet which receives drops from the squeeze bottle; and
a cross member attached to the axial member, said cross member having a nose bridge rest and at least a first leg extending laterally from the axial member, wherein the leg has an outlet which releases drops into the eye when the rest is positioned over the nose bridge.

2. An attachment as in claim 1, wherein the cross member includes a second leg and has an arcuate shape with two symmetric legs, wherein the nose bridge rest is disposed between the legs.

3. An attachment as in claim 2, wherein the first laterally extending leg is shorter than the second leg.

4. An attachment as in claim 1, wherein the outlet terminates in a nozzle and wherein the nozzle is protected within a recess having an opening of an end of the laterally extending leg.

5. An attachment as in claim 1, wherein the axial member is adapted to removably receive the squeeze bottle.

6. An attachment as in claim 4, wherein the nozzle is oriented to release the drop directly into the eye when the axial member is held generally vertically against the nose bridge.

7. An attachment as in claim 4, wherein the recess has a cut-out disposed on an inner side to reduce contact between the edge of the recess and the drop as it is released.

8. An attachment as in claim 7, wherein the recess has at least two cut-outs.

9. An attachment as in claim 4, wherein the recess has a cut-out which allows the drop from the nozzle to pass directly to the eye when the rest is positioned vertically on the nose bridge.

10. An attachment as in claim 4, wherein the nozzle is oriented to release the drop onto an edge of the recess when the axial member is held generally vertically against the nose bridge.

11. A system for dispensing droplets, said system comprising:
an attachment as in claim 1; and
a squeeze bottle attached to deliver drops to the attachment.

12. A method for dispensing drops into an eye of a patient, said method comprising:
aligning a squeeze bottle axially with a bridge of a patient's nose;
dispensing a drop in a generally axial direction toward the bridge; and
laterally channeling the drop so that it is dropped into an eye of the patient.

13. A method as in claim 12, wherein the drop is channeled into an inner corner of the eye.

14. A method as in claim 12, wherein aligning comprises resting a cross member attached to the squeeze bottle on the patient's nose bridge.

15. A method as in claim 14, wherein laterally channeling comprises diverting the drop through a lumen in the cross member having a nozzle which releases the drop disposed over the eye.

16. A method as in claim 12, wherein the nozzle is disposed directly over the eye when the dope is released so that the drop does not hit an edge of the recess.

17. A method as in claim 16, wherein an edge of the recess has at least one cut-out to reduce the risk of interfering with the released drop and/or to vent the region over the eye while the cross-member is in place.

18. A method as in claim 12, wherein the nozzle is disposed to release the drop onto an edge of the recess so that even if the patient's face is not perfectly horizontal, the drop will be caught by, and directed to the medial canthal or inner corner by the flared cross member or chute.
